(19) United States
(12) Patent Application Publication
(10) Pub. No.: US 2002/0048603 A1
(43) Pub. Date: Apr. 25, 2002

(54) Hydrogel composition

(76) Inventors: Fred Burmeister, Atlantic Highlands, NJ (US); Thomas E. Carroll, South Belmar, NJ (US)

Correspondence Address: MOSER, PATTERSON & SHERIDAN L.L.P., 595 SHREWSBURY AVE, FIRST FLOOR, SHREWSBURY, NJ, 07702, US

(21) Appl. No.: 10/007,930

(22) Filed: Dec. 7, 2001

(51) Int. Cl. A61K 9/14

(52) U.S. Cl. 424/486; 424/488

(57) ABSTRACT

A hydrogel composition and method of forming such composition suitable as a cosmetic composition for application to skin or hair is provided. The hydrogel composition comprises two or more gelling agents in an aqueous solution. The two or more gelling agents are hydrogen bonded so as to create a co-polymer network in the aqueous solution. The hydrogel composition may also include additives such as cross-linking agents, hydrophilic additive and lipophilic additives to modify the properties of the composition.

### BACKGROUND OF THE INVENTION

1. Field of the Invention

The present invention relates to hydrogel compositions and, more particularly to polysaccharide-based hydrogel compositions.

2. Description of the Background Art

Hydrogel compositions typically comprise gelling agents in an aqueous solution. In general, the gelling agents may include synthetic polymers such as, for example, polyacrylic acid and polyvinyl alcohol and/or natural polymers such gelatin and a variety of polysaccharides. Hydrogel compositions may be used in a variety of consumer or personal care products such as, air fresheners, antiperspirants, and/or skincare creams and lotions.

For example, U.S. Pat. No. 6,180,122 discusses a hydrogel composition including a polysaccharide, Gellan gum, blended with at least one hydrocolloid to form a cosmetic composition useful for skin, scalp and or hair applications. Additionally, U.S. Pat. No. 6,071,506 discusses a hydrogel composition including a polysaccharide gum dispersed with a cross-linking agent to form a gel composition useful as an air freshener.

One problem encountered with some hydrogel compositions is that the gelling agent used typically determines the physical properties of films formed therefrom. For example, hydrogel compositions based on polysaccharides such as gellan gum typically exhibit physical properties associated with such polysaccharides to produce films that are brittle or friable. Likewise, hydrogel compositions based on synthetic polymers such as polyvinyl alcohol typically exhibit physical properties associated with such synthetic polymers to produce hard, brittle films. Hydrogel compositions that produce hard, brittle films are undesirable for some applications (e.g., cosmetic compositions) because they may crack and peel off the skin, thus being ineffective in the process of skin moisturization.

Thus, there is a need to form hydrogel compositions that overcome the drawbacks listed above.

### SUMMARY OF THE INVENTION

A hydrogel composition and method of forming such composition suitable as a cosmetic composition for application to skin, nails or hair is provided. The hydrogel composition comprises two or more gelling agents in an aqueous solution. The two or more gelling agents are hydrogen bonded so as to create a co-polymer network in the aqueous solution. The hydrogel composition may also include additives such as cross-linking agents, hydrophilic additives and lipophilic additives, to modify the properties of the composition.

### DETAILED DESCRIPTION

A hydrogel composition and method of forming such composition is provided. The hydrogel composition comprises two or more gelling agents in an aqueous solution.

The two or more gelling agents may include polysaccharides and/or synthetic polymers. Suitable polysaccharides may include Gellan gum, Xantham gum, and gelatin, among others. Suitable synthetic polymers include polyvinyl alcohol, polyvinyl pyrollidone, carbomer and polyvinyl acetate, among others.

The two or more gelling agents are hydrogen bonded so as to create a co-polymer network in the aqueous solution. The co-polymer network is formed by heating the aqueous solution including the one or more gelling agents to a temperature sufficient that hydrogen bonds are formed. The hydrogen bonds may be formed at varying temperatures depending on the particular gelling agents used. Generally, hydrogen-bonding occurs when the aqueous solution is heated to a temperature greater than about 60° C.

The two or more gelling agents may form intermolecular hydrogen bonds with each other. Intramolecular hydrogen bonds may also be formed. The extent of the hydrogen bonding in the aqueous solution determines the viscosity of the hydrogel composition formed therefrom. The viscosity may range from that of a pourable liquid to a thick immobile syrup.

In addition to the temperature, the time period that the aqueous solution is heated determines the extent to which the gelling agents react to form the hydrogen bonds and varies depending on the composition of the aqueous solution. Depending on the temperature, some hydrogen bonds may be formed after about 1-2 minutes, while others may take 30-60 minutes.

The hydrogel composition may also include additives such as cross-linking agents, hydrophilic additives and lipophilic additives, to modify the properties of the composition.

The cross-linking agents may be used to cross-link the hydrogen-bonded polymer chains to create a water insoluble, harder film. Suitable cross-linking agents may include calcium chloride (CaCl₂), MgCl₂ and AlCl₃, among others.

Suitable hydrophilic additives may include proteins, amino acids, humectants, alpha hydroxy acids, beta hydroxy acids, herbal extracts, yeast extracts, dyes, fragrance, protein hydrolysates, yeast and yeast derivatives (mineral complexes and vitamin complexes), water soluble, vitamins and co-factors, enzymes, superoxide dismutase, glandular extracts, glycols, glycosaminoglycans, rubifacients, allantoin/allantoin derivatives, cellulosic polymers, guar gums, gum acacia and other nautral gums, PVP and its copolymer resins, acrylic polymers/coplymers/crosspolymers, ethoxylated fatty alcohols, ethoxylated lanolin, ethylene oxide polymers and block polymers.

Examples of suitable humectants include propylene glycol, glycerin, sorbitol, sugars (fructose/sucrose/lactose), honey and its derivatives, starch and starch derivatives, protein (polypetides, peptides), amino acids and urea.

Lipophilic agents may be included in the hydrogel to improve lubrication during the application to the skin (rub-in). These materials also act as a barrier to water loss. Suitable lipophilic agents may include lanolin, lanolin derivatives, mineral oil and other hydrocarbon oils, petrolatum, squalane, isopropyl myristate, isopropyl palmitate, isopropyl lanolate, myristyl myristate, C12-15 alcohols benzoate, vegetable oil triglycerides, castor oil, isostearyl isostearate, vegelatum, triglycerides, animal fat triglycerides, vegetable waxes jojoba, etc.), silicone “oils”, silicone waxes, silicone polymers, fluorocarbon oils and polymers, fatty alcohols, fatty alcohol ethoxylates, propoxylates, fatty alcohol esters, herbal oils, glandular oil extracts, fragrance and flavor oils, menthol, menthol derivatives, hormones, oil soluble vitamins and antioxidants.

### EXAMPLE I

A skin formulation in accordance with the invention was prepared by combining the following ingredients;

| INGREDIENT | AMOUNT (weight %) |
|---|---|
| Demineralized Water | qs to 100 |
| Gellan Gum | 0.01 to 5 |
| Polyvinyl alcohol | 0.01 to 10 |
| Preservative | qs |
| Propylene glycol | 0.01 to 10 |
| Glycerin | 0.01 to 10 |
| CaCl₂ (1% solution) | 0.00 to 0.50 |
| Color, Fragrance | qs |

The Gellan gum and polyvinyl alcohol were dispersed in the demineralized water. The solution was heated at about 60° C. to about 90° C. for up to about one hour as required to form hydrogen bonds. The variation of the percentage of Gellan gum determines the fluidity and hardness of the hydrogel. The variation of the percentage of the polyvinyl alcohol imparts resistance to mechanical stress, plasticity and alters the surface adhesive properties thereof.

After the hydrogen bonds are formed, the preservative, propylene glycol and glycerin are added and the solution is mixed to dissolve them therein. The propylene glycol and glycerin are humectants which plasticize the hydrogel composition and aid in the delivery and retention of moisture to the skin.

The mixture is cooled to approximately 70° C. and CaCl₂ is added thereto, as required. The CaCl₂ is a cross-linking agent that cross-links the reacted polymer chains and creates a water-insoluble, harder film. Thereafter, color and/or fragrance additives may be added as desired.

### EXAMPLE II

A skin formulation in accordance with the invention was prepared by combining the following ingredients;

| INGREDIENT | AMOUNT (weight %) |
|---|---|
| Demineralized Water | qs to 100 |
| Gellan Gum | 0.01 to 5 |
| Polyvinyl alcohol | 0.01 to 10 |
| Polyvinyl Pyrrolidone | 0.01 to 10 |
| Preservative | qs |
| CaCl₂ (1% solution) | 0.00 to 0.50 |

The Gellan gum, polyvinyl alcohol and polyvinyl pyrrolidone were dispersed in the demineralized water. The solution was heated at about 60°C. to about 90° C. for up to about one hour as required to form hydrogen bonds. The variation of the percentage of Gellan gum determines the fluidity and hardness of the hydrogel. The variation of the percentage of the polyvinyl alcohol imparts resistance to mechanical stress, plasticity and alters the surface adhesive properties thereof. The variation of the percentage of the polyvinyl pyrrolidone imparts surface “tack” to enhance adhesion to the skin as well as acting as a skin conditioning, irritation reducing agent.

After the hydrogen bonds are formed, the preservative is added and the solution is mixed to dissolve it therein. The mixture is cooled to approximately 70° C. and CaCl₂ is added thereto, as required. The CaCl₂ is a cross-linking agent that cross-links the reacted polymer chains and creates a water-insoluble, harder film. Thereafter, color, fragrance and/or humectants such as propylene glycol and glycerin additives may be added as desired.

### EXAMPLE III

A skin formulation in accordance with the invention was prepared by combining the following ingredients;

| INGREDIENT | AMOUNT (weight %) |
|---|---|
| Demineralized Water | qs to 100 |
| Gellan Gum | 0.01 to 5 |
| Polyvinyl alcohol | 0.01 to 10 |
| Polyvinyl Pyrrolidone | 0.01 to 10 |
| Carbomer | 0.01 to 1 |
| Preservative | qs |
| Triethanolamine | qs |
| CaCl₂ (1% solution) | 0.00 to 0.50 |

The carbomer was dispersed in one-half of the total amount of demineralized water and the pH of the dispersion is adjusted to a pH of about 5.5 or higher with an appropriate neutralizing agent such as, for example triethanolamine.

The Gellan gum, polyvinyl alcohol and polyvinyl pyrrolidone were dispersed in the remaining one-half of the total amount of demineralized water. This solution was heated at about 60° C. to about 90° C. for up to about one hour as required to form hydrogen bonds. The variation of the percentage of Gellan gum determines the fluidity and hardness of the hydrogel. The variation of the percentage of the polyvinyl alcohol imparts resistance to mechanical stress, plasticity and alters the surface adhesive properties thereof. The variation of the percentage of the polyvinyl pyrrolidone imparts surface “tack” to enhance adhesion to the skin as well as acting as a skin conditioning, irritation reducing agent.

After the hydrogen bonds are formed, the mixture is cooled to about 70° C. and the preservative or other additives are dissolved therein. The carbomer mixture is then added to this solution and mixed therewith while maintaining the temperature thereof at about 70° C. The variation of the percentage of the carbomer “blocks” the formation of a cast film, resulting in a fluid hydrogel composition.

Thereafter, CaCl₂ is added to the mixture. CaCl₂ is a cross-linking agent that cross-links the reacted polymer chains and creates a water-insoluble, harder film.

Although several preferred embodiments, which incorporate the teachings of the present invention have been shown and described in detail, those skilled in the art can readily devise many other varied embodiments that still incorporate these teachings.

What is claimed is:

1. A hydrogel composition, comprising:
two or more gelling agents in an aqueous solution, wherein the two or more gelling agents are hydrogen-bonded to create a co-polymer network in the aqueous solution.

2. The hydrogel composition of claim 1 wherein at least one of the two or more gelling agents are intramolecularly hydrogen-bonded.

3. The hydrogel composition of claim 1 wherein the two or more gelling agents are intermolecularly hydrogen-bonded.

4. The hydrogel composition of claim 1 further comprising a cross-linking agent.

5. The hydrogel composition of claim 4 wherein the cross-linking agent forms a water-insoluble film.

6. The hydrogel composition of claim 1 further comprising a hydrophilic additive.

7. The hydrogel composition of claim 1 further comprising a lipophilic additive.

8. The hydrogel composition of claim 1 wherein the at least one of the two or more gelling agents is a polysaccharide.

9. The hydrogel composition of claim 8 wherein the polysaccharide is selected from the group consisting of Gellan gum, Xantham gum, and gelatin.

10. The hydrogel composition of claim 1 wherein the at least one of the two or more gelling agents is a synthetic polymer.

11. The hydrogel composition of claim 10 wherein the synthetic polymer is selected from the group consisting of polyvinyl alcohol, polyvinyl pyrollidone, carbomer and polyvinyl acetate.

12. The hydrogel composition of claim 4 wherein the cross-linking agent is selected from the group consisting of calcium chloride (CaCl₂), MgCl₂ and AlCl₃.

13. The hydrogel composition of claim 6 wherein the hydrophilic agent is selected from the group consisting of proteins, amino acids, humectants, alpha hydroxy acids, beta hydroxy acids, herbal extracts, yeast extracts, dyes, fragrance, protein hydrolysates, yeast and yeast derivatives (mineral complexes and vitamin complexes), water soluble, vitamins and co-factors, enzymes, superoxide dismutase, glandular extracts, glycols, glycosaminoglycans, rubifacients, allantoin/allantoin derivatives, cellulosic polymers, guar gums, gum acacia and other nautral gums, PVP and its copolymer resins, acrylic polymers/coplymers/crosspolymers, ethoxylated fatty alcohols, ethoxylated lanolin, ethylene oxide polymers and block polymers.

14. The hydrogel composition of claim 13 wherein the humectant is selected from the group consisting of propylene glycol, glycerin, sorbitol, sugars (fructose/sucrose/lactose), honey and its derivatives, starch and starch derivatives, protein (polypetides, peptides), amino acids and urea.

15. The hydrogel composition of claim 7 wherein the lipophilic agent is selected from the group consisting of lanolin, lanolin derivatives, mineral oil and other hydrocarbon oils, petrolatum, squalane, isopropyl myristate, isopropyl palmitate, isopropyl lanolate, myristyl myristate, C12-15 alcohols benzoate, vegetable oil triglycerides, castor oil, isostearyl isostearate, vegelatum, triglycerides, animal fat triglycerides, vegetable waxes (jojoba, etc.), silicone “oils”, silicone waxes, silicone polymers, fluorocarbon oils and polymers, fatty alcohols, fatty alcohol ethoxylates, propoxylates, fatty alcohol esters, herbal oils, glandular oil extracts, fragrance and flavor oils, menthol, menthol derivatives, hormones, oil soluble vitamins and antioxidants.

16. A cosmetic composition for application to skin or hair, comprising:
two or more gelling agents in a aqueous solution, wherein the two or more gelling agents are hydrogen-bonded to create a co-polymer network in the aqueous solution.

17. The cosmetic composition of claim 16 wherein at least one of the two or more gelling agents are intramolecularly hydrogen-bonded.

18. The cosmetic composition of claim 16 wherein the two or more gelling agents are intermolecularly hydrogen-bonded.

19. The cosmetic composition of claim 16 further comprising a cross-linking agent.

20. The cosmetic composition of claim 19 wherein the cross-linking agent forms a water-insoluable film.

21. The cosmetic composition of claim 16 further comprising a hydrophilic additive.

22. The cosmetic composition of claim 16 further comprising a lipophilic additive.

23. The cosmetic composition of claim 16 wherein at least one of the two or more gelling agents is a polysaccharide.

24. The cosmetic composition of claim 23 wherein the polysaccharide is selected from the group consisting of Gellan gum, Xantham gum, and gelatin.

25. The cosmetic composition of claim 16 wherein the at least one of the two or more gelling agents is a synthetic polymer.

26. The cosmetic composition of claim 25 wherein the synthetic polymer is selected from the group consisting of polyvinyl alcohol, polyvinyl pyrollidone and polyvinyl acetate.

27. The cosmetic composition of claim 19 wherein the cross-linking agent is selected from the group consisting of calcium chloride (CaCl₂), MgCl₂ and AlCl₃.

28. The cosmetic composition of claim 21 wherein the hydrophilic agent is selected from the group consisting of proteins, amino acids, humectants, alpha hydroxy acids, beta hydroxy acids, herbal extracts, yeast extracts, dyes, fragrance, protein hydrolysates, yeast and yeast derivatives (mineral complexes and vitamin complexes), water soluble, vitamins and co-factors, enzymes, superoxide dismutase, glandular extracts, glycols, glycosaminoglycans, rubifacients, allantoin/allantoin derivatives, cellulosic polymers, guar gums, gum acacia and other nautral gums, PVP and its copolymer resins, acrylic polymers/coplymers/crosspolymers, ethoxylated fatty alcohols, ethoxylated lanolin, ethylene oxide polymers and block polymers.

29. The cosmetic composition of claim 28 wherein the humectant is selected from the group consisting of propylene glycol, glycerin, sorbitol, sugars (fructose/sucrose/lactose), honey and its derivatives, starch and starch derivatives, protein (polypetides, peptides), amino acids and urea.

30. The cosmetic composition of claim 22 wherein the lipophilic agent is selected from the group consisting of lanolin, lanolin derivatives, mineral oil and other hydrocarbon oils, petrolatum, squalane, isopropyl myristate, isopropyl palmitate, isopropyl lanolate, myristyl myristate, C12-15 alcohols benzoate, vegetable oil triglycerides, castor oil, isostearyl isostearate, vegelatum, triglycerides, animal fat triglycerides, vegetable waxes (jojoba, etc.), silicone “oils”, silicone waxes, silicone polymers, fluorocarbon oils and polymers, fatty alcohols, fatty alcohol ethoxylates, propoxylates, fatty alcohol esters, herbal oils, glandular oil extracts, fragrance and flavor oils, menthol, menthol derivatives, hormones, oil soluble vitamins and antioxidants.

31. A method of forming a hydrogel composition, comprising:
heating an aqueous solution comprising two or more gelling agents to form a hydrogen-bonded co-polymer network in the aqueous solution.

32. A method of forming a cosmetic composition for application to skin or hair, comprising:
heating an aqueous solution comprising two or more gelling agents to form a hydrogen-bonded co-polymer network in the aqueous solution.
